(19) United States
(12) Patent Application Publication
(10) Pub. No.: US 2006/0004107 A1
(43) Pub. Date: Jan. 5, 2006

(54) Method for designing a pharmaceutical compound specific to a desired receptor, a designed compound thereby, and pharmaceutical compositions containing the same

(76) Inventor: Masanori Ojima, Osaka (JP)

Correspondence Address: HARNESS, DICKEY & PIERCE, P.L.C., P.O.BOX 8910, RESTON, VA, 20195, US

(21) Appl. No.: 10/882,330

(22) Filed: Jul. 2, 2004

(51) Int. Cl. A61K 31/557; G06F 19/00

(52) U.S. Cl. 514/573; 702/19

(57) ABSTRACT

A method for designing a pharmaceutical compound specific to a receptor that can induce vasodilatation and/or inhibit platelet aggregation, a compound designed thereby and pharmaceutically acceptable salts thereof, and to their pharmaceutical compositions containing the same, whose side effect is decreased. The method is useful to design a structure of a pharmaceutical compound used for inducing vasodilatation and/or inhibiting platelets aggregation. The designed compound is useful for inhibiting thrombosis formation and/or for treating ischemic diseases.

### BACKGROUND OF THE INVENTION

1. Field of the Invention

The present invention relates to a method for designing a pharmaceutical compound specific to a desired receptor, a compound designed thereby and pharmaceutically acceptable salts thereof and to their pharmaceutical compositions containing the same as an active ingredient. Particularly, the invention relates to a method for designing a pharmaceutical compound specific to a certain desired pharmaceutical receptor, a compound designed thereby and pharmaceutically acceptable salts thereof, and to their pharmaceutical compositions containing the same, whose side effect is decreased. More particularly, the invention relates to a method for designing a pharmaceutical compound specific to a receptor that can induce vasodilatation and/or inhibit platelet aggregation, a compound designed thereby and pharmaceutically acceptable salts thereof, and to their pharmaceutical compositions containing the same, whose side effect is decreased.

Pharmaceutical compounds designed according to the method for designing a pharmaceutical compound of the invention comprises, prostaglandin derivatives, 9-hydroxy 10-trans,12-cis-octadecadienoic acid (9-HODE) derivatives, a phenyl hexane-1,4-dione derivative and an adenosine derivative.

2. Description of the Related Art

It is known that medicines express their pharmaceutical effects by binding to their receptors on or in cells and subsequent transduction of signals to the cells.

Generally, even when one kind of medicine is administered to an organism, if the molecules of the medicine bind to plural receptors in the organism, plural effects occur. Therefore, when the molecules of the medicine bind also to an undesirable receptor, an expected desirable pharmaceutical effect is accompanied by undesirable effects (side effects). For example, some prostaglandin compounds have a desirable pharmaceutical effect such as inhibitory activity of platelet aggregation and/or vasodilatation activity, but also have undesirable side effects (e.g. inflammation). This is because a pharmaceutical molecule may have several conformations in situ using intramolecular hydrogen bonds thereof and then one conformation may bind to the desirable receptor and other conformations may bind to the undesirable receptors to cause side effects. Separation of side effects from a targeting effect is one of the most important issues in the pharmaceutical field.

For this reason, lots of researches in pharmaceutical receptors have been done by lots of researchers to design and then prepare more effective and more specific pharmaceuticals. A study to find out necessary and sufficient conditions for a target receptor, such as charge distributions in the receptor to which a pharmaceutical molecule binds, distances of the charges and a pocket-like space of the receptors based on the structure-activity relationships, is one of approaches to design a medicine specific for targeted effects.

In the specification of the application, a figure of a receptor made of the necessary and sufficient conditions such as charge distributions in the receptor to which a pharmaceutical molecule binds, distance of said charges and space of the receptors, is referred to as a receptor model.

For instance, receptor models for muscaric receptor involved in contraction of smooth muscle were reported (A. H. Beckett. et al., J. Pharm. Pharmcol., Vol. 15, p. 362-371, 1963; A. Bebbington et al., Adv. Drug Res., vol. 2, p. 143-172, 1965; J. M. Schlman et al., J. Med. Chem., vol. 26, p. 817-823, 1983).

According to the above reports, a muscaric receptor has two positive charges and one negative charge in the receptor, wherein distances between the negative charge and the two positive charges are about 3 Angstrom (Å) and in a range of 5-7 Angstrom (Å), respectively.

Prostaglandins are widely used and their various useful biological activities are known. For example, prostaglandin I2 and prostaglandin E1 have inhibitory activity of platelet aggregation, vasodilative activity and so on. Therefore, prostaglandins and their derivatives are used or expected to be clinically useful for arteriosclerosis obliterans, thromboangiitis obliterans, (i.e. Buerger disease), intermittent claudication and the like.

On the other hand, prostaglandins have side effects such as inflammation, which disturb safety clinical use.

For example, prostaglandin E1, D2 and I2 bind to receptors which both inhibit and induce platelet aggregation (B. Ashby, Mol. Pharmacol. vol. 36, p. 866-873, 1989).

Regarding derivatives of prostaglandins, lots of derivatives are disclosed. For example, intramolecular lactones of prostaglandins, 9-dehydroxy or 9-deoxy prostaglandins are disclosed in some literatures (E. J. Corey et al., J. Am. Chem. Soc., vol. 97, p. 653-654, 1975; G. L. Bundy et al., J. Med. Chem., vol. 26, p. 790-799, 1983; G. L. Bundy et al., J. Med. Chem., vol. 26, p. 1090-1099, 1983.). However the distance of the charges in the above intramolecular lactones does not seem to have been taken into consideration.

In order to design a pharmaceutical compound specific to a receptor that can induce vasodilatation and/or inhibit platelet aggregation, the inventor has made effort to find out a selective receptor model for inhibitory activity of platelets aggregation (anti-aggregation activity of platelets) and for vasodilatation activity in a mammalian body.

The inventor has researched in structure-activity relationships between the activities and the structures of the compounds as well as the intramolecular hydrogen bonds of the compounds. For this purpose, compounds that induce/inhibit vasodilatation and compounds that induce/inhibit platelet aggregation, such as muscarine, muscarine analogs, acetyl choline, acetyl choline analogs, adrenaline, adrenaline analogs, isoproterenol, prostaglandin E1, prostaglandin I2, prostaglandin D2, adenosine, 9-hydroxy-10-trtans-12-cis-octadienoic acid (9-HODE) etc., have been used. Information about isoproterenol, adenosine and 9-HODE can be obtained from literatures (For adenosine and isoproterenol, D. C. B. Mills et al., Biochem. J., vol. 121, p. 185-196, 1971; for 9-HODE, D. Y. Henry et al., Er. J. Biochem., vol. 170, p. 389-394, 1987.). The structure-activity relationships are estimated using HGS molecular model, taking accounts both holdings and non-holding of intramolecular hydrogen bond. Charges in said each molecule are determined by the method of CNDO/2 (V. Kothekar et al., Int. J. Quantum Chem., vol. 20, p. 167-178, 1981.), MNDO(S. Miyamoto et al,. Chem. Pharm. Bull., vol. 35, p. 4510-4516, 1987) and the Huckel's extension method (J. R. Hoyland et al., J. Med. Chem., vol. 15, p. 84-86, 1971.).

As a result, the inventor has found out a receptor model specific to anti-aggregation of platelets and vasodilatation, as illustrated in FIG. 1. The receptor model comprises a pocket-like space defined by a major axis having a length of about 14 Angstrom (Å) and a minor axis having a length of about 12.5 Angstrom (Å), wherein said space has one positive charge 1 and two negative charges (a first negative charge 2 and a second negative charge 3), wherein a distance between the positive charge 1 and the first negative charge 2 is in a range of 4.2-4.9 Angstrom (Å), a distance between the positive charge 1 and the second negative charge 3 is in a range of 6.9-8.1 Angstrom (Å) and a distance between the first negative charge 2 and the second negative charge 3 is about 5.2 Angstrom (Å), and wherein a projection lies near halfway between the positive charge 1 and the second negative charge 3.

Also it has been found that a more preferable receptor model for a compound to induce vasodilatation is a receptor wherein the charges distance between the positive charge 1 and the first negative charge 2 is about 4.2 Angstrom (Å), a distance between the positive charge 1 and the second negative charge 3 is about 6.9 Angstrom (Å), and a distance between the first negative charge 2 and the second negative charge 3 is about 5.2 Angstrom (Å).

In addition, it has been found that a more preferable receptor model for a compound to inhibit aggregation of platelets is a receptor wherein the charges distance between the positive charge 1 and the first negative charge 2 is about 4.9 Angstrom (Å), a distance between the positive charge 1 and the second negative charge 3 is about 8.1 Angstrom (Å), and a distance between the first negative charge 2 and the second negative charge 3 is about 5.2 Angstrom (Å).

In the process of research in receptor models for the anti-aggregation and the vasodilatation, receptor models for aggregation of platelets (FIG. 2) and for ileum contraction/inflammation (FIG. 3) which are different from the receptor model for anti-aggregation and vasodilative activities, are also found out, respectively by, the present inventor. The receptor model receptor for aggregation of platelets has a pocket like space whose major axis and minor axis have a length of about 12 Angstrom (Å) and a length of about 11 Angstrom (Å), respectively. The receptor models for ileum contraction/inflammation has a pocket like space whose major axis and minor axis have a length of about 11.2 Angstrom (Å) and a length of about 10.5 Angstrom (Å), respectively. Both the receptor model for aggregation of platelets and the receptor model for ileum contraction/inflammation have two positive (a first positive charge and a second positive charge) and one negative charge in the space of the receptor, respectively. In addition, the distances of the charges of the receptor models are different from those of the receptor model for anti-aggregation of platelets and vasodilatation. That is, in the receptor models for aggregation of platelets (FIG. 2), a distance between the negative charge and the first positive charge is about 7.6 Angstrom (Å), a distance between the negative charge and the second positive charge is about 3.2 Angstrom (Å), and a distance between the two positive charges is about 6.1 Angstrom (Å). In the receptor models for ileum contraction/inflammation (FIG. 3), a distance between the negative charge and the first positive charge is about 5.8 Angstrom (Å), a distance between the negative charge and the second positive charge is about 3 Angstrom (Å), and a distance between the two positive charges is about 4.5 Angstrom (Å).

Further, it has been found that a receptor model for inducing vasocontraction is different from the receptor model for inducing vasodilatation in the charge distributions and distances between the charges. The receptor model for inducing vasocontraction has two positive charges (first positive charge and second positive charge) and a negative charge. In the receptor, the distance between the first positive charge and the second positive charge is about 6.0 Angstrom (Å), a distance between the first positive charge and the negative charge is about 8.2 Angstrom (Å), and a distance between the second positive charge and the negative charge is about 3.2 Angstrom (Å).

By designing the compound capable of fitting the receptor model specific to anti-aggregation of platelets and vasodilative receptors, the present inventor has succeeded in obtaining compounds with specific activity of anti-aggregation of platelets and vasodilatation.

### SUMMARY OF THE INVENTION

Main objects of the invention are to provide a method for designing a pharmaceutical compound specific to a receptor that can induce vasodilatation and/or inhibit platelet aggregation, a compound designed thereby and pharmaceutically acceptable salts thereof, and to their pharmaceutical compositions containing the same. A further object of the invention is to provide prostaglandin derivatives, 9-hydroxy 10-trans, 12-cis octadecadienoic acid (9-HODE) derivatives, a phenylhexane-dione derivative and an adenosine derivative, which are designed according to the designing method of the invention and having useful activity with low side effects, and to provide pharmaceutical compositions thereof. These objects and advantages of the invention will become apparent to persons skilled in the art from the following description.

The invention provides a method for designing a pharmaceutical compound specific to a receptor capable of inducing vasodilatation or inhibiting platelet aggregation, comprising:
designing a pharmaceutical molecule capable of fitting a receptor model comprising a pocket-like space defined by a major axis having a length of about 14 Angstrom (Å) and minor axis having a length of about 12.5 Angstrom (Å), wherein said space has a positive charge, a first negative charge and a second negative charge, wherein a distance between the positive charge and the first negative charge is in a range of 4.2-4.9 Angstrom (Å), a distance between the positive charge and the second negative charge is in a range of 6.9-8.1 Angstrom (Å), and a distance between the first negative charge and the second negative charge is about 5.2 Angstrom (Å).

Furthermore, the invention provides a compound designed by the method for designing a pharmaceutical compound mentioned above.

Furthermore, in the invention, it is preferable that the designed compound is a prostaglandin derivative of the formula [I]: 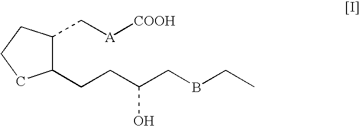
wherein A is —(CH₂)₅—, —CH₂CH₂CH₂—CH═CH—, —CH₂—O—(CH₂)₃—, CH₂—O—CH₂—CH═CH—, —CH═CH—(CH₂)₃— or —CO—(CH₂)₄—; B is —CH₂CH₂—, —CH═CH—, —CH(OH)—CH₂— or —CH₂CH(OH)—; and C is —CO— or —CH(α—OH)—; or a pharmaceutically acceptable salt thereof, with proviso that A is neither —(CH₂)₅— nor —CO—(CH₂)₄— when B is —CH₂CH₂— and C is —CH(α—OH)— at the same time.

Furthermore, in the invention, the prostaglandin derivative of the formula [I] wherein C is —CH(α—OH)—, or a pharmaceutically acceptable salt thereof is preferable.

Furthermore, in the invention, the prostaglandin derivative of the formula [I] wherein C is —CO—, or a pharmaceutically acceptable salt thereof is preferable.

Furthermore, in the invention, it is preferable that the designed compound is a carbacyclin derivative selected from the group consisting of 13,14-dihydro-carbacyclin and 13,14-dihydro-isocarbacyclin, or a pharmaceutically acceptable salt thereof.

Furthermore, in the invention, it is preferable that the designed compound is 13,14-dihydro-9,11-epoxymethano-prostaglandin H2 or a pharmaceutically acceptable salt thereof.

Furthermore, in the invention, it is preferable that the designed compound is a prostaglandin E 1,11-lactone derivative of the formula [II] 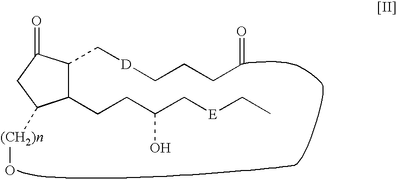
wherein D is —CH₂O— or —COCH₂—; E is —CH₂CH₂— or —CH═CH—; and n is an integer of 0-2.

Furthermore, in the invention, it is preferable that the designed compound is a prostaglandin E 1,15-lactone derivative of the formula [III] 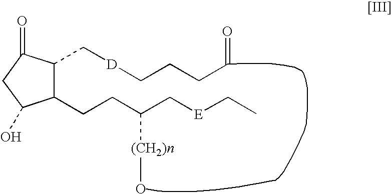
wherein D is —CH₂O— or —COCH₂—; E is —CH₂CH₂— or —CH═CH—; and n is an integer of 0-2.

Furthermore, in the invention, it is preferable that the designed compound is a 9,11-epoxymethano-prostaglandin H 1,15-lactone derivative of the formula [IV] 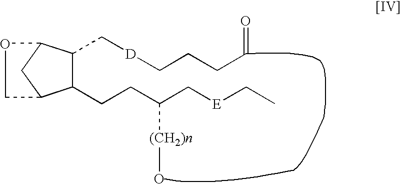
wherein D is —CH₂O— or —COCH₂—; E is —CH₂CH₂— or —CH═CH—; and n is an integer of 0-2.

Furthermore, in the invention, it is preferable that the designed compound is a prostaglandin D 1,9-lactone derivative of the formula [V] 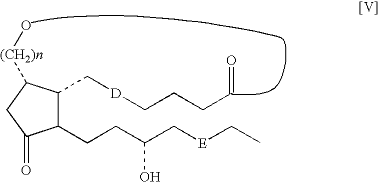
wherein D is —CH₂O— or —COCH₂—; E is —CH₂CH₂— or —CH═CH—; and n is an integer of 0-2.

Furthermore, in the invention, it is preferable that the designed compound is a prostaglandin D 1,5-lactone derivative of the formula [VI] 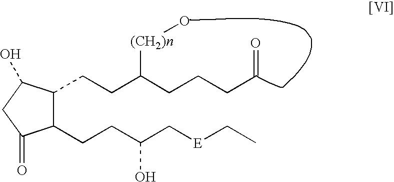
wherein E is —CH₂CH₂— or —CH═CH—; and n is an integer of 0-2.

Furthermore, in the invention, it is preferable that the designed compound is a 9,11-epoxymethano-prostaglandin H 1,5-lactone derivative of the formula [VII] 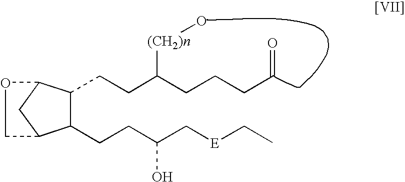
wherein E is —CH₂CH₂— or —CH═CH—; and n is an integer of 0-2.

Furthermore, in the invention, it is preferable that the designed compound is a prostaglandin D/E 1,18-lactone derivative of the formula [VIII] 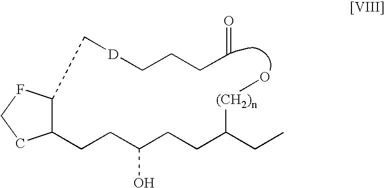
wherein D is —CH₂O— or —COCH₂—; C and F are independently —C(OH)— or —CO—, and n is an integer of 0-2 with proviso that C and F are not —C(OH)— or —CO— at the same time.

Furthermore, in the invention, it is preferable that the designed compound is a carbacyclin lactone derivative selected from the group consisting of 5,6-dihydro-13,14-dihydro-11-hydroxy carbacyclin 1,11-lactone, 5,6-dihydro-13,14-dihydro-11-dehydroxy-11-hydroxymethyl carbacyclin 1,11-hydroxymethyl-lactone, 5,6-dihydro-13,14-dihydro-11-dehydroxy-11-hydroxyethyl carbacyclin 1,11-hydroxyethyl-lactone, 5,6-dihydro-13,14-dihydro-17,18-dehydro-11-hydroxy carbacyclin 1,11-lactone, 5,6-dihydro-13,14-dihydro-17,18-dehydro-11-dehydroxy-11-hydroxymethyl carbacyclin 1,11-hydroxymethyl-lactone, 5,6-dihydro-13,14-dihydro-17,18-dehydro-11-dehydroxy-11-hydroxyethyl carbacyclin 1,11-hydroxyethyl-lactone, 5,6-dihydro-13,14-dihydro-5-hydroxymethyl carbacyclin 1,5-hydroxymethyl-lactone, 5,6-dihydro-13,14-dihydro-5-hydroxyethyl-carbacyclin 1,5-hydroxyethyl-lactone and 5,6-dihydro-13,14-dihydro-18(R)-hydroxymethyl-carbacyclin 1,18-hydroxymethyl-lactone.

Furthermore, in the invention, it is preferable that the designed compound is a 9-hydroxy 10-trans, 12-cis octadecadienoic acid (9-HODE) derivative selected from the group consisting of 9,12-dihydroxy 10-trans-octadecenoic acid 1,12-lactone, and 9,13-dihydroxy 10-trans-octadecenoic acid.

Furthermore, in the invention, it is preferable that the designed compound is 1-(3-hydroxyphenyl)-5-methylhexane-1,4-dione.

Furthermore, in the invention, it is preferable that the designed compound is a adenosine derivative of a formula [IX] 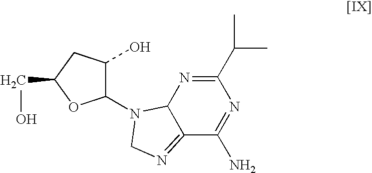
or a pharmaceutically acceptable salt thereof.

Furthermore, in the invention, it is preferable that the prostaglandin derivative is a 9-deoxy-prostaglandin E1 derivative selected from the group consisting of 9-deoxy-13,14-dihydro-5-oxa-prostaglandin E1, 9-deoxy-13,14-dihydro-17(R)-hydroxy-5-oxa-prostaglandin E1, and 9-deoxy-13,14-dihydro-18(R)-hydroxy-5-oxa-prostaglandin E1, or a pharmaceutically acceptable salt thereof.

Furthermore, in the invention, it is preferable that the prostaglandin derivative is 9-deoxy-13,14-dihydro-18(R)-hydroxy-5-oxa-prostaglandin E1, or a pharmaceutically acceptable salt thereof.

Furthermore, in the invention, it is preferable that the prostaglandin derivative is 9-deoxy-13,14-dihydro-5-oxa-prostaglandin E1, or a pharmaceutically acceptable salt thereof.

Furthermore, in the invention, it is preferable that the prostaglandin derivative is 9-deoxy-13,14-dihydro-5,6-dihydro-prostaglandin E3,9-deoxy-13,14-dihydro-5,6-dihydro-5-oxa-prostaglandin E3, or a pharmaceutically acceptable salt thereof.

Furthermore, in the invention, it is preferable that the prostaglandin derivative is 9-deoxy-13,14-dihydro-5,6-dihydro-5-oxa-prostaglandin E3 or a pharmaceutically acceptable salt thereof.

Furthermore, in the invention, it is preferable that the prostaglandin derivative is 9-dehydroxy-13,14-dihydro-18(R)-hydroxy-prostaglandin D2,9-dehydroxy-13,14-dihydro-prostaglandin D3 or a pharmaceutically acceptable salt thereof.

Furthermore, in the invention, it is preferable that the prostaglandin derivative is 9-dehydroxy-13,14-dihydro-18(R)-hydroxy-prostaglandin D2 or a pharmaceutically acceptable salt thereof.

Furthermore, in the invention, it is preferable that in the formula [III], D is —CH₂O— or —COCH₂—, E is —CH₂CH₂— and n is 2.

Furthermore, in the invention, it is preferable that in the formula [VIII], C is —CH(α—OH)—, D is —CH₂O— or —COCH₂—, F is —CO— and n is 2.

Furthermore, in the invention, it is preferable that the prostaglandin E 1,15-lactone derivative is 13,14-dihydro-15-dehydroxy-15-hydroxyethyl-5-oxa-prostaglandin E1 1,15-hydroxyethyl-lactone.

Furthermore, the invention provides a pharmaceutical composition for inhibiting thrombosis formation comprising:
a compound designed by the method for designing a pharmaceutical compound mentioned above or a pharmaceutically acceptable salt thereof, and a pharmaceutically acceptable carrier.

Furthermore, the invention provides a pharmaceutical composition for treating ischemic diseases comprising:
a compound designed by the method for designing a pharmaceutical compound mentioned above or a pharmaceutically acceptable salt thereof, and a pharmaceutically acceptable carrier.

### BRIEF DESCRIPTION OF THE DRAWINGS

Other and further objects, features, and advantages of the invention will be more explicit from the following detailed description taken with reference to the drawings wherein:

FIG. 1 is a plan view showing a receptor model for anti-aggregation of platelets and/or vasodilatation.

FIG. 2 is a plan view showing a receptor model for aggregation of platelets.

FIG. 3 is a plan view showing a receptor model for ileum contraction/inflammation.

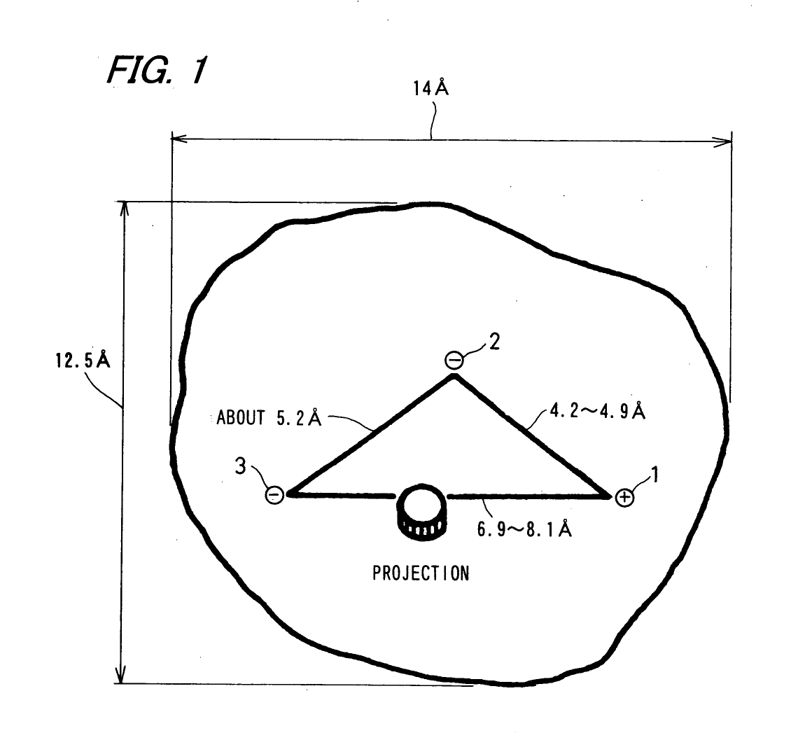

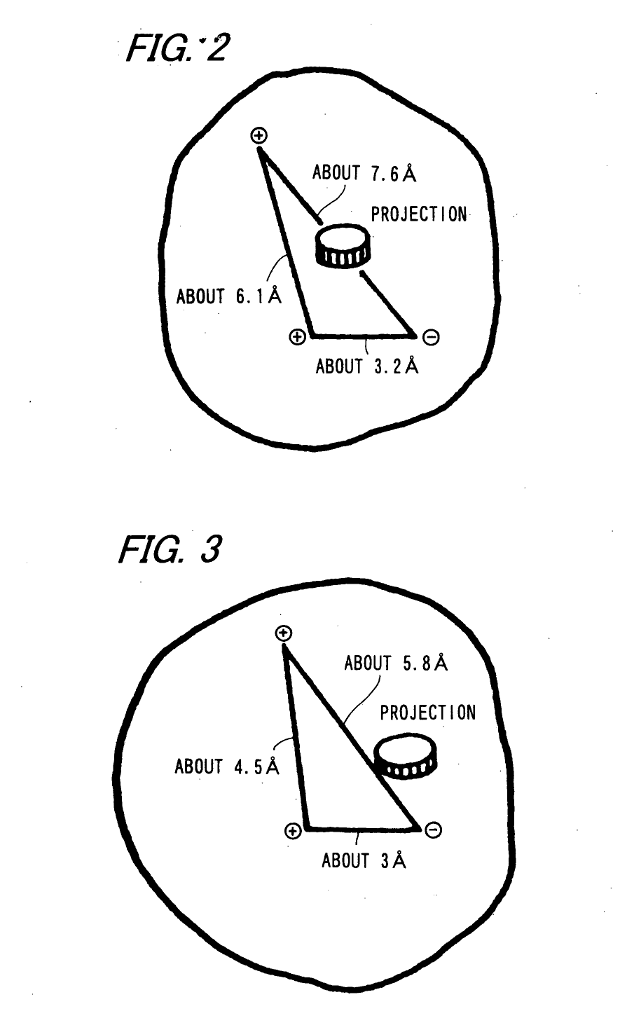

### DETAILED DESCRIPTION

Now referring to the drawings, preferred embodiments of the invention are described below.

To design the pharmaceutical molecule capable of fitting a receptor model specifically inducing vasodilatation and/or inhibiting platelet aggregation, the molecular size is designed to be able to enter a pocket-like space defined by a major axis having a length of about 14 Angstrom (Å), a minor axis having a length of about 12.5 Angstrom (Å) and a projection lying about halfway between the positive charge 1 and the second negative charge 3 of the receptor model, and the molecule is designed so that the molecule can bind to the positive charge 1, the first negative charge 2 and the second negative charge 3 in the pocket-like space, wherein a distance between the positive charge 1 and the first negative charge 2 is in a range of 4.2-4.9 Angstrom (Å), a distance between the positive charge 1 and the second negative charge 3 is in a range of 6.9-8.1 Angstrom (Å), and a distance between the first negative charge 2 and the second negative charge 3 is about 5.2 Angstrom (Å). Thus, a length of a major axis and a minor axis of the molecule to be designed are within about 14 Angstrom (Å) and about 12.5 Angstrom (Å), respectively.

The molecule size can be calculated based on the each bond length known by the person in the art (CPK model, Robert A. Harte, American Society of Biological Chemist Inc. Bethesda, Md.).

For making the molecule to bind to the three charges (the positive charge 1, the first negative charge 2, the second negative charge 3) in the pocket-like space, the charge of the molecule are designed to have a negative charge, a first positive charge as well as a second positive charge in the molecule, wherein a distance between the negative charge of the molecule and the first positive charge of the molecule is in a range of 4.2-4.9 Angstrom (Å), a distance between the negative charge and the second positive charge of the molecule is in a range of 6.9-8.1 Angstrom (Å), and a distance between the first positive charge and the second positive charge of the molecule is about 5.2 Angstrom (Å).

The charges in the molecule to be designed can be determined by the method of CNDO/2 (V. Kothekar et al., Int. J. Quantum Chem., vol. 20, p. 167-178, 1981.).

The distances of the three charges in the molecule to be designed can be calculated by means of a molecular structure model (HGS molecular structure model B Set for organic chemistry, Maruzen) or according to the literatures (L. N. Ferguston, The modern structural theory of organic chemistry, Prentice-Hall Inc., Englewood Gifts, N.J., 1963; K. Ezumi et al., J. Med. Chem., vol. 33, p. 1117-1122, 1990.).

For designing a pharmaceutical molecule capable of fitting a receptor model specifically inducing vasodilatation, it is preferable that the molecule is designed so as to bind to the positive charge 1, the first negative charge 2 as well as the second negative charge 3 in the pocket-like space of the receptor model, wherein the distance between the positive charge 1 and the first negative charge 2 is about 4.2 Angstrom (Å), a distance between the positive charge 1 and the second negative charge 3 is about 6.9 Angstrom (Å), and a distance between the first negative charge 2 and the second negative charge 3 is about 5.2 Angstrom (Å).

The compound designed by the method for designing a pharmaceutical compound mentioned above includes the following compounds (1)-(7), which are referred to as the compounds of the invention.
(1) A prostaglandin derivative of the formula [I]: 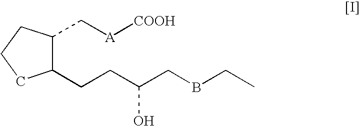
   wherein A is —(CH₂)₅—, —CH₂CH₂CH₂—CH═CH—, —CH₂—O—(CH₂)₃—, CH₂—O—CH₂—CH═CH—, —CH═CH—(CH₂)₃— or —CO—(CH₂)₄—; B is —CH₂CH₂—, —CH═CH—, —CH(OH)—CH₂— or —CH₂CH(OH)—; and C is —CO— or —CH(α—OH)—; with proviso that A is neither —(CH₂)₅-nor —CO—(CH₂)₄— when B is —CH₂CH₂— and C is —CH(α—OH)— at the same time, and a pharmaceutically acceptable salt thereof.
(2) A carbacyclin derivative selected from the group consisting of 13,14-dihydro-carbacyclin and 13,14-dihydro-isocarbacyclin, and a pharmaceutically acceptable salt thereof.
(3) 13,14-dihydro-9,11-epoxymethano-prostaglandin H2 and a pharmaceutically acceptable salt thereof.
(4) A prostaglandin derivative with an intramolecular lactone bond selected from the group consisting of the following lactones (4-1)-(4-8):
(4-1) A prostaglandin E 1,11-lactone derivative of the formula [II] 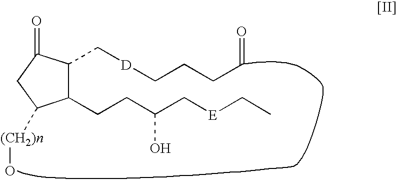
   wherein D is —CH₂O— or —COCH₂—; E is —CH₂CH₂— or —CH═CH—; and n is an integer of 0-2;
(4-2) A prostaglandin E 1,15-lactone derivative of the formula [III] 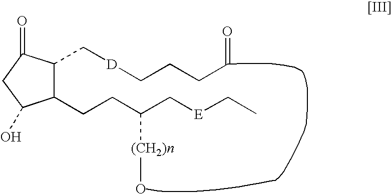
   wherein D is —CH₂O— or —COCH₂—; E is —CH₂CH₂— or —CH═CH—; and n is an integer of 0-2;
(4-3) A 9,11-epoxymethano-prostaglandin H 1,15-lactone derivative of the formula [IV] 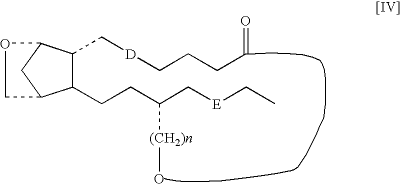
   wherein D is —CH₂O— or —COCH₂—; E is —CH₂CH₂— or —CH═CH—; and n is an integer of 0-2;
(4-4) A prostaglandin D 1,9-lactone derivative of the formula [V] 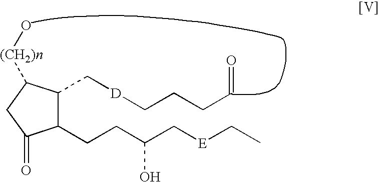
   wherein D is —CH₂O— or —COCH₂—; E is —CH₂CH₂— or —CH═CH—; and n is an integer of 0-2;
(4-5) A prostaglandin D 1,5-lactone derivative of the formula [VI] 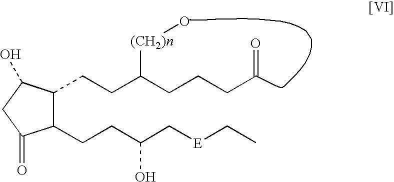
   wherein E is —CH₂CH₂— or —CH═CH—; and n is an integer of 0-2;
(4-6) A 9,11-epoxymethano-prostaglandin H 1,5-lactone derivative of the formula [VII] 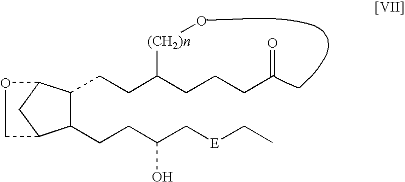
   wherein E is —CH₂CH₂— or —CH═CH—; and n is an integer of 0-2;
(4-7) A prostaglandin D/E 1,18-lactone derivative of the formula [VIII] 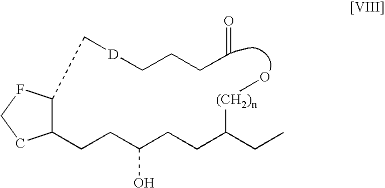
   wherein D is —CH₂O— or —COCH₂—; C and F are independently —C(OH)— or —CO—, and n is an integer of 0-2, with proviso that C and F are not —C(OH)— or —CO— at the same time;
(4-8) A carbacyclin lactone derivative selected from the group consisting of 5,6-dihydro-13,14-dihydro-11-hydroxy carbacyclin 1,11-lactone, 5,6-dihydro-13,14-dihydro-11-dehydroxy-11-hydroxymethyl carbacyclin 1,11-hydroxymethyl-lactone, 5,6-dihydro-13,14-dihydro-11-dehydroxy-11-hydroxyethyl carbacyclin 1,11-hydroxyethyl-lactone, 5,6-dihydro-13,14-dihydro-17,18-dehydro-11-hydroxy carbacyclin 1,11-lactone, 5,6-dihydro-13,14-dihydro-17,18-dehydro-11-dehydroxy-11-hydroxymethyl carbacyclin 1,11-hydroxymethyl-lactone, 5,6-dihydro-13,14-dihydro-17,18-dehydro-11-dehydroxy-11-hydroxyethyl carbacyclin 1,11-hydroxyethyl-lactone, 5,6-dihydro-13,14-dihydro-5-hydroxymethyl carbacyclin 1,5-hydroxymethyl-lactone, 5,6-dihydro-13,14-dihydro-5-hydroxyethyl-carbacyclin 1,5-hydroxyethyl-lactone and 5,6-dihydro-13,14-dihydro-18(R)-hydroxymethyl-carbacyclin 1,18-hydroxymethyl-lactone.
(5) A 9-hydroxy-10-trans-12-cis-octadecadienoic acid (9-HODE) derivative selected from the group consisting of 9, 12-dihydroxy-10-trans-octadecenoic acid 1,12-lactone and 9, 13-dihydroxy-10-trans-octadecenoic acid and a pharmaceutical acceptable salt thereof.
(6) A phenylhexane-dione derivative, that is 1-(3-hydroxyphenyl)-5-methylhexane-1,4-dione, which is designed based on the structure of isoproterenol.
(7) An adenosine derivative of the formula [IX] and a pharmaceutical salt there of.

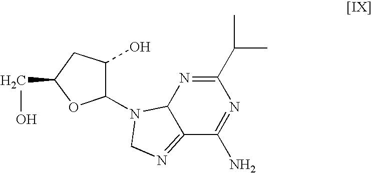

The compounds of the formula [I] may have stereoisomers depending upon configurations of a vinylene group (—CH═CH—) of A and B as well as a hydroxy group of B in the formula [I], respectively. Not only these isomers alone, but also a mixture thereof is included in the scope of the invention. A preferable configuration of hydroxy group of B is (R), a preferable configuration of a vinylene group (—CH═CH—) of —CH═CH—(CH₂)₃— in A is cis, those of vinylene groups (—CH═CH—) of —CH₂CH₂CH₂—CH═CH— and —CH₂—O—CH₂—CH═CH— in A are trans, and that of B is cis.

The compound represented by the formula [I], the above carbacyclin derivative and the above 9,11-epoxymethano-prostaglandin H2 derivative can form a pharmaceutically acceptable salt at its carboxy group.

The preferred pharmaceutically acceptable salt includes those formed with inorganic base salts formed with an alkaline metal (e.g., sodium, potassium) or alkaline earth metal (e.g., magnesium, calcium) as well as salts formed with an organic base (e.g., amines such as dicyclohexylamine, basic amino acids, such as arginine, histidine and lysine) at the carboxy group.

Hereinafter, the compounds of the formula [I] and the salts thereof are referred to as the “compound [I]”, and prostaglandin derivatives with an intramolecular lactone (4-1)-(4-8) shown above are referred to as “prostaglandin lactones of the invention”. And hereinafter, the compound [I] and the prostaglandin lactone of the invention, the carbacyclin derivative, 9,11-epoxymethano-prostaglandin H2 dedivative and the pharmaceutically acceptable salts thereof of are referred to as “prostaglandin compounds of the invention”.

The compound [I] includes 9-deoxy-prostaglandin E1 derivatives, such as 9-deoxy-13,14-dihydro-prostaglandin E1, 9-deoxy-13, 14-dihydro-Δ²-trans-prostaglandin E1, 9-deoxy-13,14-dihydro-5-oxa-prostaglandin E1, 9-deoxy-13,14-dihydro-17(R)-hydroxy-prostaglandin E1, 9-deoxy-13,14-dihydro-5-oxa-Δ²-trans-prostaglandin E1, 9-deoxy-13,14-dihydro-18(R)-hydroxy-prostaglandin E1, 9-deoxy-13,14-dihydro-17(R)-hydroxy-5-oxa-prostaglandin E1, 9-deoxy-13,14-dihydro-18(R)-hydroxy-5-oxa-prostaglandin E1, 6-keto-9-deoxy-13, 14-dihydro-Δ²-trans-prostaglandin E1, 6-keto-9-deoxy-13,14-dihydro-17(R)-hydroxy-prostaglandin E1, 6-keto-9-deoxy-13,14-dihydro-18(R)-hydroxy-5-prostaglandin E1 and pharmaceutically acceptable salts thereof.

Among these 9-deoxy-prostaglandin E1 derivatives, 5-oxa-prostagranzine E1 derivatives such as 9-deoxy-13,14-dihydro-5-oxa-prostaglandin E1, 9-deoxy-13,14-dihydro-17(R)-hydroxy-5-oxa-prostaglandin E1 and 9-deoxy-13,14-dihydro-18(R)-hydroxy-5-oxa-prostaglandin E1 are preferable. These compounds are stronger and have more specific affinity with the receptor to induce anti-aggregation of platelets and with the receptor to induce vasodilatation, while have lower affinities to the receptors to induce platelets aggregation as well as to the receptor to induce inflammation. Therefore these compounds have more potent anti-aggregation activity and more potent vasodilative activity without side effects.

The compound [I] also includes 9-deoxy-prostaglandin E3 derivatives, such as 9-deoxy-13,14-dihydro-5,6-dihydro-prostaglandin E3,9-deoxy-13,14-dihydro-prostaglandin E3, 9-deoxy-13,14-dihydro-5,6-dihydro-5-oxa-prostaglandin E3, 6-keto-9-deoxy-13,14-dihydro-5,6-dihydro-prostaglandin E3 and pharmaceutically acceptable salts thereof. Among these 9-deoxy-prostaglandin E3 derivatives, 9-deoxy-13,14-dihydro-5,6-dihydro-prostaglandin E3 and 9-deoxy-13,14-dihydro-5,6-dihydro-5-oxa-prostaglandin E3 are preferable, and 9-deoxy-13,14-dihydro-5,6-dihydro-5-oxa-prostaglandin E3 is the most preferable especially from view point of decreased side effect.

The compound [I] further includes 9-dehydroxy-prostaglandin D derivatives, such as 9-dehydroxy-13,14-dihydro-prostaglandin D2,9-dehydroxy-13,14-dihydro-18(R)-hydroxy-prostaglandin D2,9-dehydroxy-13,14-dihydro-prostaglandin D3 and pharmaceutically acceptable salts thereof. Among these prostaglandin D derivatives, 9-dehydroxy-13,14-dihydro-18(R)-hydroxy-prostaglandin D2, 9-dehydroxy-13,14-dihydro-prostaglandin D3 are preferable, and 9-dehydroxy-13,14-dihydro-18(R)-hydroxy-prostaglandin D2 is the most preferable.

The prostaglandin E 1,11-lactone derivative of the formula [II] includes 13,14-dihydro-prostaglandin E1 1,11-lactone, 13,14-dihydro-11-dehydroxy-11-hyroxymetyl-prostaglandin E1 1,11-hydroxymethyl-lactone (i.e. a compound formed by lactonization between 1-carboxyl group and 11-hydroxylmethyl group. In this specification, a lactone compound of the prostaglandin derivative with lactone bond formed between 1-carboxyl group and a branched hydroxyalkyl group is named in this way), 13,14-dihydro-11-dehydroxy-11-hyroxyethyl-prostaglandin E1 1,11-hydroxyethyl-lactone, 13,14-dihydro-5-oxa-prostaglandin E1 1,11-lactone, 13,14-dihydro-11-dehydroxy-11-hyroxymethyl-5-oxa-prostaglandin E1 1,11-hydroxymethyl-lactone, 13,14-dihydro-11-dehydroxy-11-hyroxyetyl-5-oxa-prostaglandin E1 1,11-hydroxyethyl-lactone, 13,14-dihydro-6-keto-prostaglandin E1 1,11-lactone, 13,14-dihydro-11-dehydroxy-11-hyroxymethyl-6-keto-prostaglandin E1 1,11-hydroxymethyl-lactone and 13,14-dihydro-11-dehydroxy-11-hyroxyethyl-6-keto-prostaglandin E1 1,11-hydroxyethyl-lactone.

Among the prostaglandin E 1,11-lactone derivative of the formula [II], 13,14-dihydro-11-dehydroxy-11-hyroxyethyl-prostaglandin E1 1,11-hydroxyethyl-lactone, 13,14-dihydro-11-dehydroxy-11-hyroxyethyl-5-oxa-prostaglandin E1 1,11-hydroxyethyl-lactone and 13,14-dihydro-11-dehydroxy-11-hyroxyethyl-6-keto-prostaglandin E1 1,11-hydroxyethyl-lactone are preferable.

The prostaglandin E 1,15-lactone derivative of the formula [III] includes, for example, 13,14-dihydro-5-oxa-prostaglandin E1 1,15-lactone, 13,14-dihydro-15-dehydroxy-15-hydroxymethyl-5-oxa-prostaglandin E1 1,15-hydroxymethyl-lactone, 13,14-dihydro-15-dehydroxy-15-hydroxyethyl-5-oxa-prostaglandin E1 1,15-hydroxyethyl-lactone, 13,14-dihydro-5-oxa-prostaglandin E3 1,15-lactone, 13,14-dihydro-15-dehydroxy-15-hydroxymethyl-5-oxa-prostaglandin E3 1,15-hydroxymethyl-lactone, 13,14-dihydro-15-dehydroxy-15-hydroxyethyl-5-oxa-prostaglandin E3 1,15-hydroxyethyl-lactone, 13,14-dihydro-6-keto-prostaglandin E1 1,15-lactone, 13,14-dihydro-15-dehydroxy-15-hydroxymethyl-6-keto-prostaglandin E1 1,15-hydroxymethyl-lactone, 13,14-dihydro-15-dehydroxy-15-hydroxyethyl-6-keto-prostaglandin E1 1,15-hydroxyethyl-lactone, 13,14-dihydro-6-keto-prostaglandin E3 1,15-lactone, 13,14-dihydro-15-dehydroxy-15-hydroxymethyl-6-keto-prostaglandin E3 1,15-hydroxymethyl-lactone, and 13,14-dihydro-15-dehydroxy-15-hydroxyethyl-6-keto-prostaglandin E3 1,15-hydroxyethyl-lactone.

Among the prostaglandin E 1,15-lactone derivative of the formula [III], 13,14-dihydro-15-dehydroxy-15-hydroxyethyl-5-oxa-prostaglandin E1 1,15-hydroxyethyl-lactone, 13,14-dihydro-15-dehydroxy-15-hydroxyethyl-5-oxa-prostaglandin E3 1,15-hydroxyethyl-lactone, 13,14-dihydro-15-dehydroxy-15-hydroxyethyl-6-keto-prostaglandin E1 1,15-hydroxyethyl-lactone and 13,14-dihydro-15-dehydroxy-15-hydroxyethyl-6-keto-prostaglandin E3 1,15-hydroxyethyl-lactone are more preferable because these compound have a high tolerance for decomposition by a prostaglandin-15-hydroxy-dehydrogenase and have more reduced side effects.

The 9,11-epoxymethano-prostaglandin H 1,15-lactone derivative of the formula [IV] includes, for example, 13,14-dihydro-9,11-epoxymethano-prostaglandin H1 1,15-lactone, 13,14-dihydro-15-dehydroxy-15-hydroxyethyl-9,11-epoxymethano-prostaglandin H2 1,15-hydroxyethyl-lactone and 13,14-dihydro-15-dehydroxy-15-hydroxyethyl-9,11-epoxymethano-prostaglandin H3 1,15-hydroxyethyl-lactone.

More preferable compound is 13,14-dihydro-15-dehydroxy-15-hydroxyethyl-9,11-epoxymethano-prostaglandin H2 1,15-hydroxyethyl-lactone.

A prostaglandin D 1,9-lactone derivative of the formula [V] includes, for example, 13,14-dihydro-prostaglandin D1 1,9-lactone, 13,14-dihydro-9-dehydroxy-9-hydroxymethyl-prostaglandin D1 1,9-hydroxymethyl-lactone, 13,14-dihydro-9-dehydroxy-9-hydroxyethyl-prostaglandin D1 1,9-hydroxyethyl-lactone, 13,14-dihydro-prostaglandin D2 1,9-lactone, 13,14-dihydro-9-dehydroxy-9-hydroxymethyl-prostaglandin D2 1,9-hydroxymethyl-lactone, 13,14-dihydro-9-dehydroxy-9-hydroxyethyl-prostaglandin D2 1,9-hydroxyethyl-lactone, 13,14-dihydro-prostaglandin D3 1,9-lactone, 13,14-dihydro-9-dehydroxy-9-hydroxymethyl-prostaglandin D3 1,9-hydroxymethyl-lactone and 13,14-dihydro-9-dehydroxy-9-hydroxyethyl-prostaglandin D3 1,9-hydroxyethyl-lactone.

Among these compounds, 13,14-dihydro-9-dehydroxy-9-hydroxyethyl-prostaglandin D1 1,9-hydroxyethyl-lactone, 13,14-dihydro-9-dehydroxy-9-hydroxyethyl-prostaglandin D2 1,9-hydroxyethyl-lactone and 13,14-dihydro-9-dehydroxy-9-hydroxyethyl-prostaglandin D3 1,9-hydroxyethyl-lactone are preferable.

The prostaglandin D 1,5-lactone derivative of the formula [VI] includes, for example, 5,6-dihydro-13,14-dihydro-5-hydroxymethyl-prostaglandin-D2,5,6-dihydro-13,14-dihydro-5-hydroxyethyl-prostaglandin-D2 1,5-hydroxyethyl-lactone, 5,6-dihydro-13,14-dihydro-5-hydroxymethyl-prostaglandin-D3 1,5-hydroxymethyl-lactone and 5,6-dihydro-13,14-dihydro-5-hydroxyethyl-prostaglandin-D3 1, 5-hydroxyethyl-lactone. Preferable prostaglandin D 1,5-lactone derivatives are 5,6-dihydro-13,14-dihydro-5-hydroxymethyl-prostaglandin-D2 and 5,6-dihydro-13,14-dihydro-5-hydroxymethyl-prostaglandin-D3.

Among 9,11-epoxymethano-prostaglandin H 1,5-lactone derivatives of the formula [VII], 5,6-dihydro-13,14-dihydro-5-hydroxymethyl-9,11-epoxymethano-prostaglandin H2 1,5-hydroxymethyl-lactone and 5,6-dihydro-13,14-dihydro-5-hydroxymethyl-9,11-epoxymethano-prostaglandin H3 1,5-hydroxymethyl-lactone are preferable.

The prostaglandin D/E 1,18-lactone derivative of the formula [VIII] includes, for example, 13,14-dihydro-17,18-dihydro-18(R)-hydroxymethyl prostaglandin E1 1,18-hydroxymethyl-lactone, 13,14-dihydro-17,18-dihydro-18(R)-hydroxymethyl prostaglandin E3 1,18-hydroxymethyl-lactone, 13,14-dihydro-17,18-dihydro-18(R)-hydroxymethyl-5-oxa-prostaglandin E3 1,18-hydroxymethyl-lactone, 13,14-dihydro-17,18-dihydro-18(R)-hydroxymethyl-6-keto-prostaglandin E3 1,18-hydroxymethyl-lactone, 13,14-dihydro-17,18-dihydro-18(R)-hydroxymethyl-5-oxa-prostaglandin D3 1,18-hydroxymethyl-lactone.

Among the all prostaglandin lactones, more preferable lactones are the prostaglandin E 1,15-lactone derivative of the formula [III] wherein D is —CH₂O— or —COCH₂—, E is —CH₂CH₂— and n is 2), and the prostaglandin E 1,18-lactone derivative of the formula [VIII] wherein C is —CH(α—OH)—, D is —CH₂O— or —COCH₂—, F is —CO— and n is 2). 13,14-dihydro-15-dehydroxy-15-hydroxyethyl-5-oxa-prostaglandin E1 1,15-hydroxyethyl-lactone (the compound of the formula [III], wherein D=—CH₂O—, E=—CH₂CH₂— and n=2) is the most preferable from the viewpoint of selective activity and the stability of the compound.

Compound [I] wherein C is —CH(OH)-(That is, 9-deoxy prostaglandin E derivatives), may be prepared, for example, from the corresponding prostaglandin E derivative as follows.

A C-9 carbonyl group of prostaglandin E derivative corresponding to 9-deoxy-prostagrangin E of the formula [I] is converted to a hydrazone, preferably, p-toluensulfonylhydrazone by reacting with a hydrazine compound such as p-toluensulfonylhydrazine, and then the hydrazone group is reductively cleaved by metal hydrides such as lithium aluminum hydride, sodium borohydride, catecholborane or sodium cyanoborohydride. The preparation of the above hydrazone and its reductive cleavage may be carried out successively as a one-pot reaction without isolation of the hydrazone.

Depending on the reaction condition of the reductive cleavage of the hydrazone, a reduction of a carboxy group at the C-1 terminal (C-1 carboxy group) of the prostaglandin derivative might be accompanied to give a C-1 alcohol. In such case, the C-1 alcohol can be selectively oxidized to a carboxy group by catalytic oxidation over a metal oxide catalyst such as platinum oxide.

Compound [I] wherein C is —CO— (That is, 9-dehydroxy prostaglandin D derivatives), may be prepared, for example, from the corresponding prostaglandin D derivative protected by protecting group (e.g. tetyrahydropyranyl or trimethyl silyl) on hydroxy group(s) (e.g. 15-hydroxy group) other than a 9-hydroxy group, as follows. The 9-hydroxy group is converted to a lower alkyl sulfate by reacting with a lower alkyl sulfonyl chloride, such as methanesulfonyl chloride in the presence of an organic amine (e.g. pyridine, triethyl amine). Then the lower alkyl sulfate is reductively cleaved by metal hydrides, for instance, lithium aluminum hydride, sodium borohydride to afford a 9-dehydroxy prostaglandin compound. Usually this reductive cleavage reaction is accompanied by a reduction reaction of a carboxy group at the C-1 terminal (C-1 carboxy group) to give a C-1 alcohol. Therefore, the C-1 alcohol is selectively oxidized to a carboxy group by catalytic oxidation over a metal oxide catalyst such as platinum oxide. Then, the hydrohxy protecting group(s) is deprotected to give a desired 9-dehydroxy prostaglandin compound of the invention.

Prostaglandins or protected prostaglandins to be used as the starting materials for preparing prostaglandin of the invention may be prepared, according to the general procedure described in literatures (e.g. R. Noyori et al., Convergent Synthesis of Prostaglandins, Advances in Prostaglandin, Thromboxane, and Leukotriene Research, vol. 15, p. 295, edited by O. Hayaishi and S. Yamamoto. Raven Press, New York, 1985).

The prostaglandin lactone of the invention may be prepared by intramoleclar lactonization with esterificatin reagent, for example, by Corey-Nicolaou lacterization procedure (E. J. Corey et al., J. Am. Chem. Soc., vol. 97, P. 653, 1975). In case of preparing the prostaglandin lactone of the invention with one or more hydroxy groups, the hydroxy group(s) which is not used for forming an intramolecular lactone bond is preferably protected by the protective group that may be deprotected in a mild condition after the lactonization reaction. Preferable protective groups are trimethylsilyl, ter-butyldimetyl silyl and tetrahydropyranyl etc. The starting material of the prostaglandin lactone of the invention may me prepared according to the literature described above.

9,12-dihydroxy-10-trans-octadecenoic acid 1,12-lactone of the invention may be prepared by lactonization of 9,12-dihydroxy-10-trans-octadecenoic acid using the lactonization method described above for the lactonization of prostaglandin compound, followed the separation of the lactonization products. 9,13-dihydroxy-10-trans-octadecenoic acid of the invention may be prepared by the selective hydroxylation at the 13-position and hydrogenation at the 12-position of 9-hydroxy-10-trans-12-cis-octadienoic acid.

The phenylhexane-dione derivatives, 1-(3-hydroxyphenyl)-5-methylhexane-1,4-dione which is designed according to the designing method of the invention based on the structure of isoproterenol, may be prepared by the conventional ketone synthesis.

An adenosine derivative of the formula [X]described above may be prepared by the coupling reaction of 2-isopropyl adenine with deoxyribose via a conventional process.

The compound of the invention can have three intramolecular charges (one negative charge, a first positive charge and a second positive charge in the molecule of the compound) in its solution as well as in a human body. The distance between the charges is such that a distance between the negative charge of the molecule and the first positive charge of the molecule is in a range of 4.2-4.9 Angstrom (Å), a distance between the negative charge and the second positive charge of the molecule is in a range of 6.9-8.1 Angstrom (Å), and a distance between the first positive charge and the second positive charge of the molecule is about 5.2 Angstrom (Å). And the size of the molecule of the compound is suitable to enter into the pocket of the receptor for anti-aggregation of platelets and/or receptor for vasodilatation.

The compound of the invention is designed not to have intramolecular three charges, two positive charges and one negative, which are necessary charges to bind receptors to induce side effects such as platelets aggregation, inflammation and ileum contraction. Therefore the compound of the invention has much more affinitive to useful targeting receptors (anti-aggregation of platelets and/or receptor for vasodilatation) than to undesirable receptors.

The specificity of the compounds of the invention to the receptors is confirmed for example, by theoretical prostaglandin receptors (M. Ojima et al., Prostaglandins Leukotriens and Essential Fatty Acids, vol. 47, p. 69-76, 1992) as well as binding assay using actual receptors (A. M. Siegl, J. Clin. Invest., vol. 63, p. 215-220, 1979; A. Botella, European Journal of Pharmacology, vol. 237, p. 131-137, 1993).

As described above, the compounds of the invention are specific to useful receptors to induce vasodilatation and/or to inhibit platelet, while have lower affinity to the receptors that cause side effects. Therefore, the compounds of the invention have specific activities of inhibitory activity of platelet aggregation and vasodilative activity.

Accordingly, the compounds of the invention have good therapeutic and preventive effects on diseases, such as thrombosis due to platelet aggregation (for example, cerebral thrombosis, arteriosclerosis obliterans, thromboangiitis obliterans (i.e. Buerger disease), intermittent claudication); ischemic diseases due to constriction of arterial vascular smooth muscle or angiohypertonia in the heart, lung, brain (e.g., cardiac infarction, cerebral apoplexy, primary pulmonary hypertension and the like.

Thus, the pharmaceutical compositions comprising the compounds of the invention are useful as medicines for inhibiting thrombosis formation and/or for treating ischemic diseases, such as an antithrombotic drug, an anti-vasoconstriction, a drug for improving circulatory system such as the heart and the like, for treating or preventing the diseases mentioned above caused by due to platelet aggregation, constriction of arterial vascular smooth muscle or angiohypertonia, and the like.

The prostaglandin compounds of the invention used for preparing the pharmaceutical compositions are preferably used in the form of cyclodextrin clathrates to increase the stability of the prostaglandin compounds. The clathrates may be prepared by mixing a prostaglandin compound dissolved in a water-soluble organic solvent with alpha-, beta- or gamma-cyclodextrin or a mixture thereof dissolved in water or in a aqueous organic solvent, heating the mixture not to exceed 70° C., cooling to 0-10° C., and then separating the resulting cyclodextrin clathrate by filtration, centrifugation or concentrating the mixture under reduced pressure.

The compounds of the invention may be orally or parenterally administered in the form of pharmaceutical compositions, for example, tablets, capsules, solutions, injection preparations, suppositories, combined with known pharmaceutically acceptable carrier.

The pharmaceutical composition for oral administration includes solid preparations such as tablets, granules, powders, fine granules and hard capsules as well as liquid preparations such as syrups and soft capsules.

Tablets, granules, powders and fine granules are prepared by mixing the compounds of the invention with conventional pharmaceutically acceptable nontoxic carriers such as lactose, starch, crystalline cellulose, magnesium stearate, talc, and the like. Hard capsules are prepared by packing the above fine granules or powders into capsules. Syrups are prepared by dissolving or suspending the compound of the invention in an aqueous solution containing white sugar, carboxymethyl cellulose and the like. Soft capsules are prepared by dissolving or suspending the compound of the invention in fatty diluents, such as vegetable oils, oil emulsions and glycols, and packing the solution or suspension into soft capsules.

The pharmaceutical composition of the invention also includes suppositories for rectal administration or vaginal administration. The suppositories may be prepared, for example, by the following procedure. A base for the suppository is melted by heating and the compound of the invention is mixed with the melt. Then, the mixture is cast into molds in pre-determined quantities and cooled to give suppositories. For the base of the suppository, a mixture of the monoglyceride, diglyceride and triglyceride of straight-chain saturated fatty acids, or solid polyethylene glycol whose melting point is 35-40° C. may be used. Among them, a hard fat with a hydroxyl value not exceeding 50 is preferably used for the preparation of the suppositories containing the prostaglandin compound of the invention. The term “hard fat” as used herein means a mixture of the monoglyceride, diglyceride and triglyceride of saturated fatty acid with a straight-chain of 8 to 18 carbon atoms. The hard fats with hydroxyl value not more than 50 are commercially available, for example, under the trade name of Witepsol H-35, H-5, H-15 and W-35 by Dynamit Nobel Co. and Nissan Pharmasol B-115 by Nippon Oil & Fats Co., Ltd.

The suppositories comprising prostaglandin compound of the invention and the hard fat with a hydroxyl value not exceeding 50 has better shelf life of the suppositories, and the bioavailability of the prostaglandin compound of the invention in the suppositories is better than that of the suppositories made of hard fat with high hydroxy value more than 50.

Preparations Pharmaceutical compositions comprising the compound of the invention for parenteral administration include sterile aqueous or non-aqueous solutions, suspensions, emulsions or sterile solid which can be dissolved in sterile water or other sterile solvent for injection immediately before use.

The aqueous solutions or suspensions may include distilled water for injection and physiological salt solution. Non-aqueous solutions or suspensions may include glycols (propylene glycol, polyethylene glycol, glycerol), plant oil (e.g. olive oil, soybean oil). Such compositions may comprise additional additives, such as preserving agents, emulsifying agents (e.g. lecithin, pharmaceutically acceptable surfactants etc), dispersing agents, stabilizing agent, pH-adjustment agent (e.g., glutamic acid, asparaginic acid, lysine, histidine, arginine, NaOH, HCl). They may be sterilized for example, by filtration through a bacteria-retaining filter, sterilizing agents, by irradiation.

The dose of the compound of the invention, is, for example, in the case of administering orally to an adult, advantageous that the compound of the invention is normally administered 1 to 3 times per day with a daily dose of about 0.01 to 30 mg/kg, preferably, 0.05 to 10 mg/kg, although it may varies depending upon a age or body weight of patient, administration route, conditions of diseases and the like. In case of administering by injection, the daily dose may be lower than that of oral dose.

As described hereinabove, according to the invention, there are provided the compounds of the invention having excellent selectivity for desired receptors that can induce vasodilatation and/or inhibit platelet aggregation, which are useful as medicines, for example, for treating thrombosis due to platelet aggregation, ischemic diseases, angiohypertonia or the like.

The following Examples further illustrate the invention in detail but are not to be construed to limit the scope of the invention.

A platelet aggregation test and an inflammatory test may be performed as bellows. Inhibitory activity of platelet Aggregation:

An optical Born-type aggregomater is employed, with fresh citrated human platelet-rich plasma (PRP) from donor, in the manner described (N. H. Andersen et al., Prostaglandins, vol. 19, p. 711-735, 1980). Optical density 90 sec. after ADP addition is the measure of aggregation employed throughout. ADP (7-10 μM final concentration) is added after 1 minute preincubation of a test compound and PRP. A full dose response curve for at least one standard (Prostaglandin E1) is obtained with each PRP specimen. Relative potency estimates from the full dose response curve comparisons are adjusted to prostaglandin E1 set as 100.

Inflammatory test method can be carried out by the method described in the literature (P. Crunkhorn, A. L. Willis, Br. J. Pharmac., vol. 41, p. 49-56, 1971).

### EXAMPLE 1

Preparation of 9-deoxy-13,14-dihydro-5-oxa-prostaglandin E1 designed by the method for designing a pharmaceutical compound according to the invention is prepared by following procedure (1)-(4).

(1) 5-oxa-prostaglandin E1 3.56 g (10 mmol) is dissolved in methanol (200 ml) and hydrogenated over Pd black catalyst (300 mg) under hydrogen atmosphere (1 atm) at room temperature over nigh. The catalyst is removed by filtration, the solvent is evaporated under reduced pressure and resulting residue is dried under reduced pressure to give 13,14-dihydro-5-oxa-prostaglandin E1.

(2) 13,14-dihydro-5-oxa-prostaglandin E1 (715 mg, 2 mmol) and p-toluenesulfonylhydrazide (372 mg, 2 mmol) is resolved in methanol in a 50-ml round-bottomed flask equipped with a magnetic stirring bar and a reflux condenser. The solution is stirred and heated at reflux for 10 minutes and allowed to cool to room temperature. The reaction mixture was condensed and dried under reduced pressure over silica gel to give crude p-toluenesulfonylhydrazone of 13,14-dihydro-5-oxa-prostaglandin E1.

(3) To a stirred suspension of 304 mg (8 mmol) of lithium aluminum hydride in 30 ml of dry ether in a 50-ml round-bottomed flask equipped with a magnetic stirring bar and a reflux condenser, is added dropwise a solution of the above crude hydrazone (ca 2 mmol) in 8 ml of ether. After 1 hour at 25° C., 2.5 ml of H₂O is added, followed by 0.5 ml of 10% aqueous KOH. After 3 hours of stirring, the mixture is powered into 10% sodium tartarate, and the layer is separated. Further ether extraction, drying over MgSO₄ and evaporation of the extract gives crude (11α, 15S)-11,15-dihydro-5-oxa-prost-1-ol, which is purified by column chromatography (silica gel 50 g; solvent, ethyl acetate/hexane=30/70 by volume) to give purified (11,15S)-11,15-dihydro-5-oxa-prost-1-ol.

(4) A suspension of 500 mg platinum oxide in 50 ml of distilled water is reduced with hydrogen. The system is flushed thoroughly with nitrogen and then oxygen. Solid NaHCO₃ (840 mg) is added and the temperature of the black suspension is raised to 60° C. To the vigorous stirred 60° C. suspension, a suspension of (11α,15S)-11,15-dihydro-5-oxa-prost-1-ol (346.5 mg, 1 mmol) in 30 ml of a mixture of water and acetone (water/acetone=4/1) is added. An additional 60 ml of 12% acetone water is added, and the mixture is stirred for 5 hours at 60° C. with oxygen bubbling through the reaction mixture via a syringe. The reaction mixture is cooled to room temperature, filtered through Cerite, concentrated to about 70 ml under reduced pressure at 30-35° C., acidified by aqueous NaHSO₄, extracted with ethyl acetate. The extract is washed with brine, dried over Na₂SO₄, concentrated to give crude 9-deoxy-13,14-dihydro-5-oxa-prostaglandin E1, which is purified by column chromatography (acid washed silica gel 40 g; solvent, ethyl acetate/hexane=35/65 by volume).

### EXAMPLE 2

Preparation of 13,14-dihydro-15-dehydroxy-15-hydroxyethyl-5-oxa-prostaglandin E1 1,15-hydroxyethyl-lactone designed by the method for designing a pharmaceutical compound according to the invention: A mixture of 13,14-dihydro-15-dehydroxy-15-hydroxyethyl-5-oxa-prostaglandin E1 386.5 mg (1 mmol), triphenylphosphine 393 mg (1.5 mmol), and 2,2′-dipyrydil disulfide 330 mg (1.5 mmol) and dry oxygen-free xylene 6 ml is stirred under nitrogen atmosphere at room temperature for 24 hours. The reaction mixture is diluted with 250 ml of xylene and heated at reflux for 2 hours. Xylene is removed at reduced pressure, the residue is partitionated between brine and ethyl acetate, and extracted. The ethyl acetate extract is washed with aqueous NaHCO₃ and brine, dried over Na₂SO₄ and concentrated under reduced pressure. The concentrate is purified by column chromatography (silica gel 80 g; solvent ethyl acetate/hexane=40/60 by volume). Fractions are monitored by thin layer chromatography, eluants are collected, the solvent is evaporated under reduced pressure, resulting residue is dried under reduced pressure to give 13,14-dihydro-15-dehydroxy-15-hydroxyethyl-5-oxa-prostaglandin E1 1,15-hydroxyethyl-lactone.

### EXAMPLE 3

Preparation of 13,14-dihydro-11-dehydroxy-11-hydroxyethyl-5-oxa-prostaglandin E1 1,11-hydroxyethyl-lactone designed by a method for designing a pharmaceutical compound according to the invention: A mixture of 13,14-dihydro-11-dehydroxy-11-hydroxyethyl-5-oxa-prostaglandin E1 773.2 mg (2 mmol), triphenylphosphine 787 mg (3 mmol), and 2,2′-dipyrydil disulfide 660 mg (3 mmol) and dry oxygen-free xylene 10 ml is stirred under nitrogen atmosphere at room temperature for 24 hours. The reaction mixture is diluted with 500 ml of xylene and heated at reflux for 2 hours. Xylene is removed undedr reduced pressure, the residue is partitionated between brine and ethyl acetate, and extracted. The ethyl acetate extract is washed with aqueous NaHCO₃ and brine, dried over Na₂SO₄ and concentrated under reduced pressure. The concentrate is purified by column chromatography (silica gel 160 g; solvent ethyl acetate/hexane=40/60 by volume). Fractions are monitored by thin layer chromatography, eluants are collected, the solvent is evaporated under reduced pressure, resulted residue is dried under reduced pressure to give 13,14-dihydro-11-dehydroxy-11-hydroxyethyl-5-oxa-prostaglandin E 1,11-hydroxyethyl-lactone.

### EXAMPLE 4

Capsules containing 9-deoxy-13,14-dihydro-5-oxa-prostaglandin E1 is prepared as follows, using a cyclodextrin clathrate of 9-deoxy-13,14-dihydro-5-oxa-prostaglandin E1.

(1) Preparation of cyclodextrin clathrate of 9-deoxy-13,14-dihydro-5-oxa-prostaglandin E1: 1 g of 9-deoxy-13,14-dihydro-5-oxa-prostaglandin E1 is dissolved in 140 ml of ethanol. To the solution, 17 g of β-cyclodextrin dissolved in 220 ml of hot water (60° C.) is added. The mixture is stirred gently at 60° C. to make a clear solution, cooled gradually to about 5° C. and is allowed to stand for overnight at 2° C. Resulting precipitates is collected by filtration, washed with 50% aqueous ethanol, dried at 20° C. under reduced pressure to give cyclodextrin clathrate of 9-deoxy-13,14-dihydro-5-oxa-prostaglandin E1. The clathrate contains about 6% of 9-deoxy-13,14-dihydro-5-oxa-prostaglandin E1.

(2) Preparation of capsules: The clathrate (8.3 parts by weight) is mixed with 67 parts by weight of lactose, 115 parts by weight of corn starch and 9.7 weigh part of magnesium stearate, and filled in hard capsules in an amount of 200 mg per capsules. Each capsule contains 0.5 mg of 9-deoxy-13,14-dihydro-5-oxa-prostaglandin E1.

### EXAMPLE 5

Preparation of suppositories containing 13,14-dihydro-15-dehydroxy-15-hydroxyethyl-5-oxa-prostaglandin E1 1,15-hydroxyethyl-lactone: 99.8 parts by weight of a hard fat with a hydroxyl value of 2.3 (Witepsol TM H-35, Dynamit Nobel Co.) is put in a stainless steel beaker and melted at 40 to 42° C. To the melt, 0.2 parts by weight of 13,14-dihydro-15-dehydroxy-15-hydroxyethyl-5-oxa-prostaglandin E1 1,15-hydroxyethyl-lactone is added and mixed well. The mixture at about 38° C. is poured in 1 g portions into spindle-shaped molds and cooled to suppositories each containing 2 mg of 13,14-dihydro-15-dehydroxy-15-hydroxyethyl-5-oxa-prostaglandin E1 1,15-hydroxyethyl-lactone.

### EXAMPLE 6

Tablets each containing 1 mg of 13,14-dihydro-15-dehydroxy-15-hydroxyethyl-5-oxa-prostaglandin E1 1,15-hydroxyethyl-lactone are prepared as follows. To a mixture of 1 part by weight of 13,14-dihydro-15-dehydroxy-15-hydroxyethyl-5-oxa-prostaglandin E1 1,15-hydroxyethyl-lactone, 76 parts by weight of corn starch and 163 parts by weight of microcrystalline cellulose are added to a solution of 3.3 parts by weight of hydroxypropyl cellulose in water (50 parts by weight) and the mixture is kneaded well. The kneaded mixture is passed through a mesh to produce granules. After drying the granules, 6.7 parts by weight of magnesium stearate is mixed with the granules and the mixture is tabletted by a conventional method to give tablets (each 250 mg weight).

The invention may be embodied in other specific forms without departing from the spirit or essential characteristics thereof. The present embodiments are therefore to be considered in all respects as illustrative and not restrictive, the scope of the invention being indicated by the appended claims rather than by the foregoing description and all changes which come within the meaning and the range of equivalency of the claims are therefore intended to be embraced therein.

What is claimed is:

1. A method for designing a pharmaceutical compound specific to a receptor capable of inducing vasodilatation or inhibiting platelet aggregation, comprising:
designing a pharmaceutical molecule capable of fitting a receptor model comprising a pocket-like space defined by a major axis having a length of about 14 Angstrom (Å) and minor axis having a length of about 12.5 Angstrom (Å), wherein said space has a positive charge, a first negative charge and a second negative charge, wherein a distance between the positive charge and the first negative charge is in a range of 4.2-4.9 Angstrom (Å), a distance between the positive charge and the second negative charge is in a range of 6.9-8.1 Angstrom (Å), and a distance between the first negative charge and the second negative charge is about 5.2 Angstrom (Å).

2. A compound designed by the method for designing a pharmaceutical compound according to claim 1.

3. The designed compound according to claim 2, which is a prostaglandin derivative of the formula [I]: 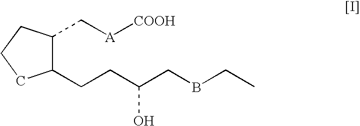
wherein A is —(CH₂)₅—, —CH₂CH₂CH₂—CH═CH—, —CH₂—O— (CH₂)₃—, —CH₂—O—CH₂—CH═CH—, —CH═CH—(CH₂)₃— or —CO—(CH₂)₄—; B is —CH₂CH₂—, —CH═CH—, —CH(OH)—CH₂— or —CH₂CH(OH)—; and C is —CO— or —CH(α—OH)—; or a pharmaceutically acceptable salt thereof, with proviso that A is neither —(CH₂)₅— nor —CO—(CH₂)₄— when B is —CH₂CH₂— and C is —CH(α—OH)— at the same time.

4. The prostaglandin derivative according to claim 3, wherein C is —CH(α—OH)—, or a pharmaceutically acceptable salt thereof.

5. The prostaglandin derivative according to claim 3, wherein C is —CO—, or a pharmaceutically acceptable salt thereof.

6. The designed compound according to claim 2, which is a carbacyclin derivative selected from the group consisting of 13,14-dihydro-carbacyclin and 13,14-dihydro-isocarbacyclin, or a pharmaceutically acceptable salt thereof.

7. The designed compound according to claim 2, which is 13,14-dihydro-9,11-epoxymethano-prostaglandin H2 or a pharmaceutically acceptable salt thereof.

8. The designed compound according to claim 2, which is a prostaglandin E 1,11-lactone derivative of the formula [II] 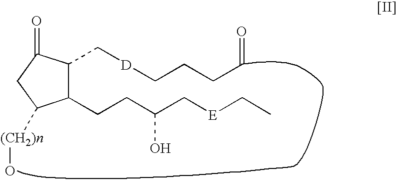
wherein D is —CH₂O— or —COCH2—; E is —CH₂CH₂— or —CH═CH—; and n is an integer of 0-2.

9. The designed compound according to claim 2, which is a prostaglandin E 1,15-lactone derivative of the formula [III] 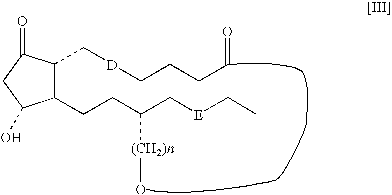
wherein D is —CH₂O— or —COCH₂—; E is —CH₂CH₂— or —CH═CH—; and n is an integer of 0-2.

10. The designed compound according to claim 2, which is a 9,11-epoxymethano-prostaglandin H 1,15-lactone derivative of the formula [IV] 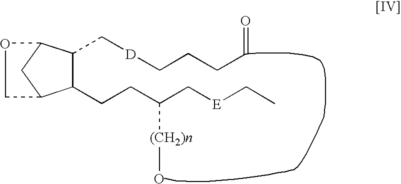
wherein D is —CH₂O— or —COCH₂—; E is —CH₂CH₂— or —CH═CH—; and n is an integer of 0-2.

11. The designed compound according to claim 2, which is a prostaglandin D 1,9-lactone derivative of the formula [V] 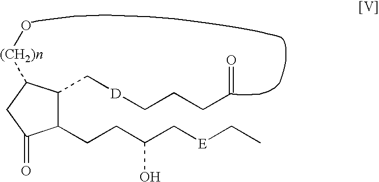
wherein D is —CH₂O— or —COCH₂—; E is —CH₂CH₂— or —CH═CH—; and n is an integer of 0-2.

12. The designed compound according to claim 2, which is a prostaglandin D 1,5-lactone derivative of the formula [VI] 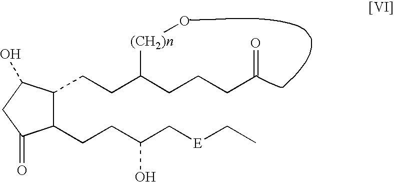
wherein E is —CH₂CH₂— or —CH═CH—; and n is an integer of 0-2.

13. The designed compound according to claim 2, which is a 9,11-epoxymethano-prostaglandin H 1,5-lactone derivative of the formula [VII] 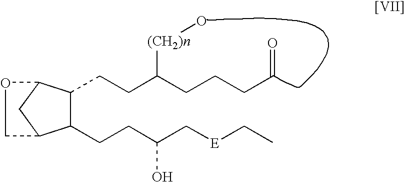
wherein E is —CH₂CH₂— or —CH═CH—; and n is an integer of 0-2.

14. The designed compound according to claim 2, which is a prostaglandin D/E 1,18-lactone derivative of the formula [VIII] 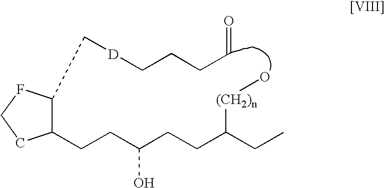
wherein D is —CH₂O— or —COCH₂—; C and F are independently —C(OH)— or —CO—, and n is an integer of 0-2 with proviso that C and F are not —C(OH)— or —CO— at the same time.

15. The designed compound according to claim 2, which is a carbacyclin lactone derivative selected from the group consisting of 5,6-dihydro-13,14-dihydro-11-hydroxy carbacyclin 1,11-lactone, 5,6-dihydro-13,14-dihydro-11-dehydroxy-11-hydroxymethyl carbacyclin 1,11-hydroxymethyl-lactone, 5,6-dihydro-13,14-dihydro-11-dehydroxy-11-hydroxyethyl carbacyclin 1,11-hydroxyethyl-lactone, 5,6-dihydro-13,14-dihydro-17,18-dehydro-11-hydroxy carbacyclin 1,11-lactone, 5,6-dihydro-13,14-dihydro-17,18-dehydro-11-dehydroxy-11-hydroxymethyl carbacyclin 1,11-hydroxymethyl-lactone, 5,6-dihydro-13,14-dihydro-17,18-dehydro-11-dehydroxy-11-hydroxyethyl carbacyclin 1,11-hydroxyethyl-lactone, 5,6-dihydro-13,14-dihydro-5-hydroxymethyl carbacyclin 1,5-hydroxymethyl-lactone, 5,6-dihydro-13,14-dihydro-5-hydroxyethyl-carbacyclin 1,5-hydroxyethyl-lactone and 5,6-dihydro-13,14-dihydro-18(R)-hydroxymethyl-carbacyclin 1,18-hydroxymethyl-lactone.

16. The designed compound according to claim 2, which is a 9-hydroxy 10-trans, 12-cis octadecadienoic acid (9-HODE) derivative selected from the group consisting of 9, 12-dihydroxy 10-trans-octadecenoic acid 1,12-lactone, and 9, 13-dihydroxy 10-trans-octadecenoic acid.

17. The designed compound according to claim 2, which is 1-(3-hydroxyphenyl)-5-methylhexane-1,4-dione.

18. The designed compound according to claim 2, which is a adenosine derivative of a formula [IX] 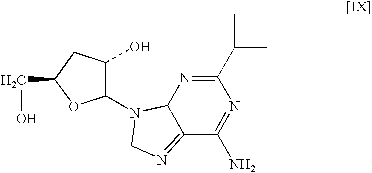
or a pharmaceutically acceptable salt thereof.

19. The prostaglandin derivative according to claim 3, which is a 9-deoxy-prostaglandin E1 derivative selected from the group consisting of 9-deoxy-13,14-dihydro-5-oxa-prostaglandin E1, 9-deoxy-13,14-dihydro-17(R)-hydroxy-5-oxa-prostaglandin E1, and 9-deoxy-13,14-dihydro-18(R)-hydroxy-5-oxa-prostaglandin E1, or a pharmaceutically acceptable salt thereof.

20. The prostaglandin derivative according to claim 3, which is 9-deoxy-13,14-dihydro-18(R)-hydroxy-5-oxa-prostaglandin E1, or a pharmaceutically acceptable salt thereof.

21. The prostaglandin derivative according to claim 3, which is 9-deoxy-13,14-dihydro-5-oxa-prostaglandin E1, or a pharmaceutically acceptable salt thereof.

22. The prostaglandin derivative according to claim 3, which is 9-deoxy-13,14-dihydro-5,6-dihydro-prostaglandin E3,9-deoxy-13,14-dihydro-5,6-dihydro-5-oxa-prostaglandin E3, or a pharmaceutically acceptable salt thereof.

23. The prostaglandin derivative according to claim 3, which is 9-deoxy-13,14-dihydro-5,6-dihydro-5-oxa-prostaglandin E3 or a pharmaceutically acceptable salt thereof.

24. The prostaglandin derivative according to claim 3, which is 9-dehydroxy-13,14-dihydro-18(R)-hydroxy-prostaglandin D2,9-dehydroxy-13,14-dihydro-prostaglandin D3 or a pharmaceutically acceptable salt thereof.

25. The prostaglandin derivative according to claim 3, which is 9-dehydroxy-13,14-dihydro-18(R)-hydroxy-prostaglandin D2 or a pharmaceutically acceptable salt thereof.

26. The prostaglandin E 1,15-lactone derivative according to claim 9, wherein D is —CH₂O— or —COCH₂—, E is —CH₂CH₂— and n is 2.

27. The prostaglandin E 1,18-lactone derivative according to claim 14, wherein C is —CH(α—OH)—, D is —CH₂O— or —COCH₂—, F is —CO— and n is 2.

28. The prostaglandin E 1,15-lactone derivative according to claim 26, which is 13,14-dihydro-15-dehydroxy-15-hydroxyethyl-5-oxa-prostaglandin E1 1,15-hydroxyethyl-lactone.

29. A pharmaceutical composition for inhibiting thrombosis formation comprising:
a compound designed by the method for designing a pharmaceutical compound according to claim 1 or a pharmaceutically acceptable salt thereof, and a pharmaceutically acceptable carrier.

30. A pharmaceutical composition for treating ischemic diseases comprising:
a compound designed by the method for designing a pharmaceutical compound according to claim 1 or a pharmaceutically acceptable salt thereof, and a pharmaceutically acceptable carrier.
